(19) United States
(12) Patent Application Publication
(10) Pub. No.: US 2011/0098315 A1
(43) Pub. Date: Apr. 28, 2011

(54) PHARMACEUTICAL COMPOSITIONS OF ROSUVASTATIN CALCIUM

(76) Inventors: Farhad Sayyad Farshi, Istanbul (TR); Recep Avci, Istanbul (TR); Serdar Apari, Istanbul (TR)

(21) Appl. No.: 12/997,248

(22) PCT Filed: Jun. 27, 2008
(86) PCT No.: PCT/IB08/52595

§ 371 Date: Dec. 9, 2010

(51) Int. Cl. A61K 31/505; A61P 3/06

(52) U.S. Cl. 514/275

(57) ABSTRACT

This invention is related to pharmaceutical compositions of rosuvastatin or pharmaceutically acceptable salts thereof especially calcium salt with sodium carbonate anhydrous which has effects on dissoltion profiles in 0.1 N HCI medium provided that using of sodium carbonate anhydrous should be presence in the range of from about 0.5% to about 2% by weight.

The present invention is related to pharmaceutical compositions of rosuvastatin or pharmaceutically acceptable salts thereof especially calcium salt with alkali or earth alkali metal-carbonate-bicarbonate or a mixture therof which have effects on dissoltion profiles in 0.1 N HCI medium provided that using of sodium carbonate anhydrous should be presence in the range of from about 0.5% to about 2% by weight of the pharmaceutical composition or molar ratio should be in the range of 1:0.43-1.75 (Rosuvastatin or its pharmaceutically acceptable salt: alkali or earth alkali metal-carbonate-bicarbonate or a mixture therof). Pharmaceutical composition is preferably oral dosage form. Oral dosage form is preferably tablet and could be other oral dosage forms such as capsule, pellet, minitablet etc.

### BACKGROUND ART

EP 521471 which is basic patent of rosuvastatin and discloses rosuvastatin as an inhibitor of HMG-CoA reductase.

In the patent of EP 0547000 it is described that formulations of HMG-CoA inhibitors with alkaline substances including sodium carbonate and/or calcium carbonate in the range of from 0.1% to 60% . However EP 0547000 does not denote that effects of the percentage or molar ratio of alkaline substances in rosuvastatin formulations on dissolution profiles in 0.1 N HCI environment. EP 0547000 points out that only effects on stability and intestinal absorbtion.

In the patent of EP 1223918 a protection is determined that using of a tribasic phosphate salt, in which the cation is multivalent, in pharmaceutical compositions of rosuvastatin. Actually, the rationale of using of tribasic phosphate salt in which multivalent cation dissolution profilesin 0.1 N HCI environment are different from other alkanizing agents. Therefore, for providing bioequivalence, if generics use alkanizing agent other than tribasic phosphate salt in which multivalent cation they could not reach desired dissolution profiles in 0.1 N HCI environment and could not achieve bioequivalance. According to information of DailyMed web site, in Crestor® tablet formulations in 5-10-20 and 40 mg, tribasic calcium phosphate is used as a tribasic phosphate salt which is included group of cation is multivalent.

EP 1905424 discloses that the percentage of alkanizing agent including sodium carbonate may vary from about 1 to about 50 by weight of the composition. EP 1905424 does not disclose effect of alkanizing agents on dissolution profiles and does not point out any percentage or molar ratio to achieve critical dissolution in 0.1 N HCI environment. Sodium carbonate per se could be used to stabilize as it is explained in that patent, but it has important effects on dissolution profiles in 0.1 N HCI environment according to our invention.

In patent application of WO 2008/062476 sodium carbonate is used as inorganic salt of monovalent cation in rosuvastatin formulations. But there is no any disclosing as to effects of using of sodium carbonate on dissolution profiles in 0.1 N HCI environment. Claim 7 discloses that using of inorganic salt of monovalent cation within the range of 0.01% to 10% by weight of the composition without denoting effects of sodium carbonate on dissolution profile.

### DETAILED DESCRIPTION OF INVENTION

In generally, dissolution environment is held by the generics as medium of pH 6.6 citrate to accomplish of bioequivalence since FDA (Food and Drug Administration) hold that dissolution environment of rosuvastatin calcium is pH 6.6 in vitro (Dissolution Methods of Drug Products). Using of alkali or earth alkali metal-carbonate-bicarbonate or a mixture of therof in any percentage in pharmaceutical composition of rosuvastatin or in any molar ratio with rosuvastatin, dissolution profiles of reference tablets and test tablets are the same or identical in pH 6.6 environment. In other words, in pH 6.6 environment, alkali or earth alkali metal-carbonate-bicarbonate or a mixture of therof in any percentage in pharmaceutical composition of rosuvastatin or in any molar ratio with rosuvastatin has no any effect on dissolution profiles and dissolution profiles are not depended on amount of alkali or earth alkali metal-carbonate-bicarbonate. In rosuvastatin tablet formulations, only holding this result is not enough to carry out bioequivalence studies and to achieve bioequivalence since reference tablet is dissolved as from time of intaking to stomach. In other words, after administration oral solid dosage form is released upon contact with stomach environment before the absorbtion. Therefore it is important to consider the dissolving in 0.1 N HCI environment. The medium of stomach is 0.1 N HCI and dissolution profiles of generic product should overlap with dissolution profiles of reference product in 0.1 N HCI environment. Thus overlapping of dissolution profiles in pH 6.6 environment alone is not sufficient. According to this invention, most probably, test product shall be bioequivalence with reference product as trademark of Crestor®.

In this application we unexpecdetly and surprisingly find out that a special molar ratio between rosuvastatin and alkali or earth alkali metal-carbonate-bicarbonate or a mixture therof provides a dissolution profile with a similarity factor greater then 50 in 0.1 N HCl dissolution media compared with Crestor®. Molar ratio is in the range of 1:0.43-1.75 (Rosuvastatin or its pharmaceutically acceptable salt: alkali or earth alkali metal-carbonate-bicarbonate or a mixture therof). Alkali metals are selected from Na or K and earth alkali metals are selected from Mg or Ca but not limited with them. Preferably sodium carbonate anhydrous

Another aspect of this invention is related to effects of alkali or earth alkali metal-carbonate-bicarbonate or a mixture therof, which are used in a specific range, on dissolution profiles of rosuvastatin calcium in medium of 0.1 N HCI. According to this invention in medium of 0.1 N HCI, dissolution profiles of rosuvastatin calcium tablets including alkali or earth alkali metal-carbonate-bicarbonate or a mixture therof, which are used in a specific range by the weight of pharmaceutical composition, are the same with reference tablets that are sold on the market with trademark of Crestor®. To overlap of dissolution profiles of reference tablet and test tablet in medium of 0.1 N HCI, alkali or earth alkali metal-carbonate-bicarbonate or a mixture of therof are used in the range of from about % 0.5 to % 2, preferably from about 0.6% to 1.4% by weight of the tablet.

In another aspect of the invention is pharmaceutical composition comprising at least one pharmaceutically suitable excipient. Excipient could be a filler or a disintegrating agent or a lubricant or another suitable excipient or mixture of thereof.

In yet another aspect of this invention is a pharmaceutical composition comprising rosuvastatin or its pharmaceutically acceptable salt includes alkali or earth alkali metal-carbonate-bicarbonate or a mixture therof characterizing in that alkali or earth alkali metal-carbonate-bicarbonate or a mixture of therof are used in the range of 0.5% to 2% by weight of the pharmaceutical composition and the active ingredient has an in vitro dissolution profile in 0.1 N HCI environment with a similarity factor (f2) of at least 50 to 100 compared to the reference dissolution profile.

The similarity factor f2 is a measurement of the similarity through a point by point comparison as shown in equation 1.

$${f}_{2} = 50 * log \frac{100}{\sqrt{1 + \frac{1}{n} {\sum }_{t = 1}^{n} {( {R}_{t} - {T}_{t} )}^{2}}} \qquad ( Equation 1 )$$

n: is the number of sampling time points

Rₜ: is the amount drug released from a reference batch at time t

Tₜ: is the amount drug released from a test batch at time t.

Generally, f₂ values greater than 50 ensure sameness of the performance of the reference product and test product.

Pharmaceutical composition is preferably oral dosage formulations such as tablet, capsule, pellet, minitablet etc. Especially preferred oral dosage formulation is tablet.

Using of alkali or earth alkali metal-carbonate-bicarbonate or a mixture therof within the range of about 0.5% to about 2% by weight of composition and/or within the molar ratio in the range of 1:0.43-1.75 (Rosuvastatin or its pharmaceutically acceptable salt: alkali or earth alkali metal-carbonate-bicarbonate or a mixture therof) also do not bring about impurities such as lactones.

It is unexpectedly and surprisingly found that using of alkali or earth alkali metal-carbonate-bicarbonate or a mixture therof in tablet formulations of rosuvastatin calcium within the range of about from 0.5% to about 2% by weight of the tablet, preferably about from 0.6% to about 1.4% and/or within the molar ratio in the range of 1:0.43-1.75 (Rosuvastatin or its pharmaceutically acceptable salt: alkali or earth alkali metal-carbonate-bicarbonate or a mixture therof), dissolution profiles in the 0.1 N HCI environment is about identical with reference tablet, known as Crestor®. In rosuvastatin calcium formulations, effects of using of alkali or earth alkali metal-carbonate-bicarbonate or a mixture therof, which is in mentioned specific ranges, its effect on dissolution profiles in 0.1 N HCI environment is unexpected result, since it is not known by the prior art and it is only known that using of alkali or earth alkali metal-carbonate-bicarbonate or a mixture therof in rosuvastatin calcium formulations, it prevents occuring impurities such aslactone.

Any sutiable dissolution method could be used to reach dissolution profiles. In this invention USP method-1 (basket) is preferred and used. However USP method-1 (basket) is not exhaustive and other eligible methods such as but not limited USP method-II (paddle) could be contemplated.

Pharmaceutical composition is preferably prepared as a tablet that exhibits a dissolution profile as less than or equal to 85% of the total amount of rosuvastatin calcium is released in 30 minutes after combining the tablet with 900 ml of a dissolution medium at 37° C.±0.5° C. through using of USP method 1 (basket) and basket speed is 100 rpm

Pharmaceutical composition has a dissolution profile as rosuvastatin calcium is released in the range of 15% to 25% in 5 minutes, 40% to 50% in 10 minutes, 60% to 70% in 15 minutes, 70% to 80% in 20 minutes, 80% to 90% in 30 minutes, 85% to 95% in 45 minutes and 90 to 100% in 60 minutes after association of 900 ml of a dissolution medium at 37° C.±0.5° C. and USP method 1 (basket) and basket speed is 100 rpm.

Alkali metal carbonates are preferably Na₂CO₃or K₂CO₃, but not limited with them.

In this invention sodium carbonate anhydrous is preferred and used. Earth alkali metals are preferably CaCO₃ or MgCO₃, but not limited with them.

### EXAMPLE 1

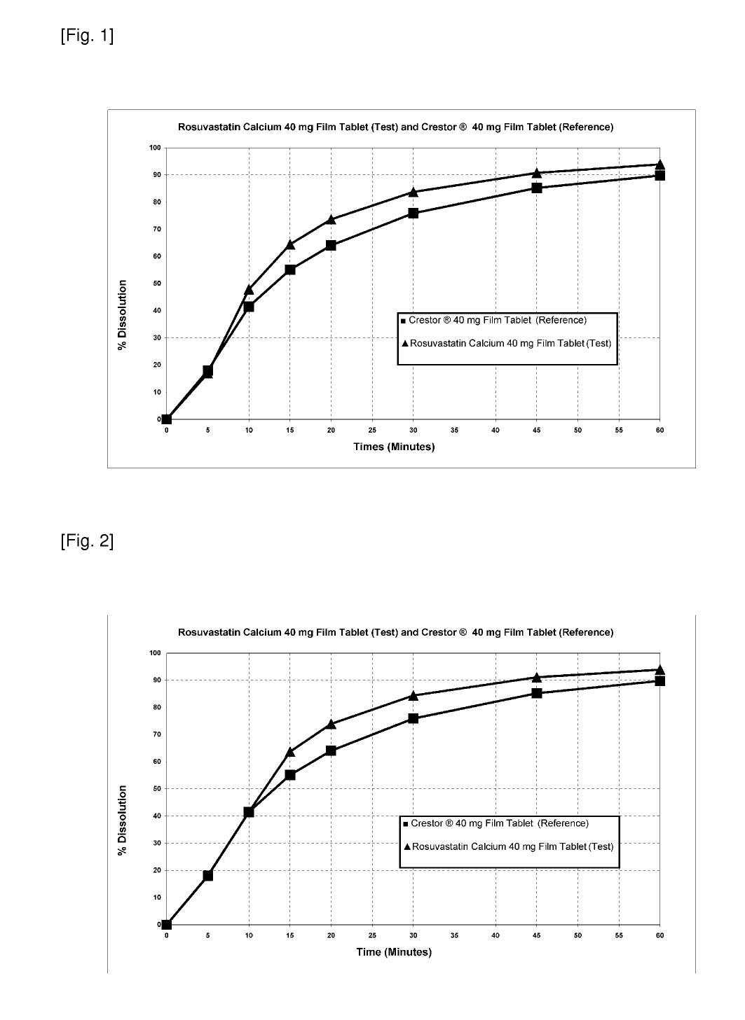

Rosuvastatin calcium test tablet includes sodium carbonate anhydrous as within the molar ratio in the range of 1:1.17 (Rosuvastatin calcium:sodium carbonate anhydrous) is released in 0.1 N HCI environment under conditions of 900 ml of a dissolution medium at 37° C.±0.5° C., USP method 1 (basket), 100 rpm basket speed wherein tablet exhibits a dissolution profile (FIG. 1 and Table 1). Under mentioned conditions f2 value is 57.9. At the same time sodium carbonate anhydrous is 1.34% by weight of tablet.

TABLE 1
Comparison of Test and Reference Tablets (molar ratio is
1:1.17 as rosuvastatin calcium:sodium carbonate anhydrous)

Dissolved %

| | Crestor ® | Rosuvastatin |
|---|---|---|
| | 40 mg Film | Calcium |
| Time | Tablet | 40 mg Film |
| (Minutes) | (Reference) | Tablet (Test) |
| 5 | 18.1 | 17.0 |
| 10 | 41.6 | 47.9 |
| 15 | 55.1 | 64.5 |
| 20 | 64.1 | 73.7 |
| 30 | 75.9 | 83.8 |
| 45 | 85.2 | 90.8 |
| 60 | 89.8 | 93.9 |

### EXAMPLE 2

Rosuvastatin calcium test tablet includes sodium carbonate anhydrous as within the molar ratio in the range of 1:1.75 (Rosuvastatin calcium:sodium carbonate anhydrous) is released in 0.1 N HCI environment under conditions of 900 ml of a dissolution medium at 37° C.±0.5° C., USP method 1 (basket), 100 rpm basket speed wherein tablet exhibits a dissolution profile (FIG. 1 and Table 1). At the same time sodium carbonate anhydrous is 2% by weight of tablet (FIG. 2 and Table 2). Under mentioned conditions f2 value is 59.1.

TABLE 2
Comparison of Test and Reference Tablets (molar ratio is
1:1.75 as rosuvastatin calcium:sodium carbonate anhydrous)

Dissolved %

| | Crestor ® | Rosuvastatin |
|---|---|---|
| | 40 mg Film | Calcium |
| Time | Tablet | 40 mg Film |
| (Minutes) | (Reference) | Tablet (Test) |
| 5 | 18.1 | 18 |
| 10 | 41.6 | 41.5 |
| 15 | 55.1 | 63.7 |
| 20 | 64.1 | 73.9 |
| 30 | 75.9 | 84.4 |
| 45 | 85.2 | 91.1 |
| 60 | 89.8 | 93.9 |

### EXAMPLE 3

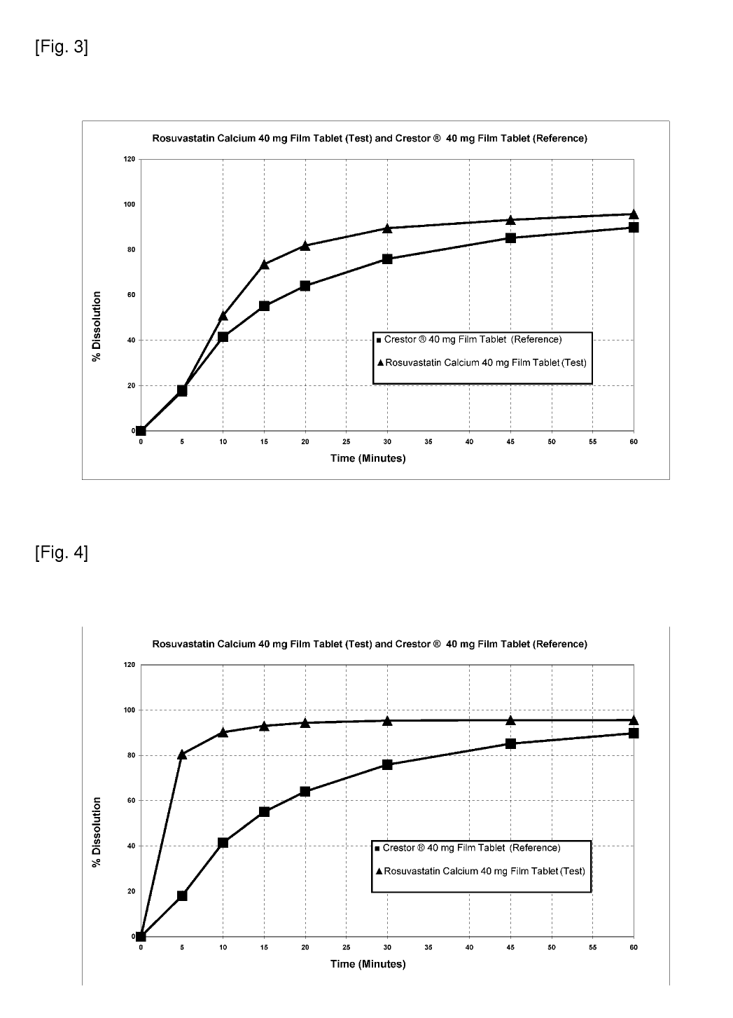

Rosuvastatin calcium test tablet includes sodium carbonate anhydrous as within the molar ratio in the range of 1:2.35 (Rosuvastatin calcium:sodium carbonate anhydrous) is released in 0.1 N HCI environment under conditions of 900 ml of a dissolution medium at 37° C.±0.5° C., USP method 1 (basket), 100 rpm basket speed wherein tablet exhibits a dissolution profile (FIG. 1 and Table 1). At the same time sodium carbonate anhydrous is 2.65% by weight of tablet (FIG. 3 and Table 3). Under mentioned conditions f2 value is 45.7.

TABLE 3
Comparison of Test and Reference Tablets (molar ratio is
1:2.35 as rosuvastatin calcium:sodium carbonate anhydrous)

Dissolved %

| | Crestor ® | Rosuvastatin |
|---|---|---|
| | 40 mg Film | Calcium |
| Time | Tablet | 40 mg Film |
| (Minutes) | (Reference) | Tablet (Test) |
| 5 | 18.1 | 17.4 |
| 10 | 41.6 | 50.9 |
| 15 | 55.1 | 73.6 |
| 20 | 64.1 | 81.9 |
| 30 | 75.9 | 89.5 |
| 45 | 85.2 | 93.2 |
| 60 | 89.8 | 95.8 |

### EXAMPLE 4

Rosuvastatin calcium test tablet includes sodium carbonate anhydrous as within the molar ratio in the range of 1:4.71 (Rosuvastatin calcium:sodium carbonate anhydrous) is released in 0.1 N HCI environment under conditions of 900 ml of a dissolution medium at 37° C.±0.5° C., USP method 1 (basket), 100 rpm basket speed wherein tablet exhibits a dissolution profile (FIG. 4 and Table 4). At the same time sodium carbonate anhydrous is 5.16% by weight of tablet. Under mentioned conditions f2 value is 22.1.

TABLE 4
Comparison of Test and Reference Tablets (molar ratio is
1:4.71 as rosuvastatin calcium:sodium carbonate anhydrous)

Dissolved %

| | Crestor ® | Rosuvastatin |
|---|---|---|
| | 40 mg Film | Calcium |
| | Tablet | 40 mg Film |
| Minutes | (Reference) | Tablet (Test) |
| 5 | 18.1 | 80.6 |
| 10 | 41.6 | 90.3 |
| 15 | 55.1 | 93.1 |
| 20 | 64.1 | 94.5 |
| 30 | 75.9 | 95.4 |
| 45 | 85.2 | 95.6 |
| 60 | 89.8 | 95.6 |

1. A pharmaceutical composition comprising rosuvastatin or its pharmaceutically acceptable salt and alkali or earth alkali metal-carbonate-bicarbonate or a mixture therof characterizing in that alkali or earth alkali metal-carbonate-bicarbonate or a mixture thereof and rosuvastatin or its pharmaceutically acceptable salt are in a molar ratio in the range of 1:0.43-1.75 (Rosuvastatin or its pharmaceutically acceptable salt: alkali or earth alkali metal-carbonate-bicarbonate or a mixture therof) to provide a dissolution profile with a similarity factor (f2) of at least 50 to 100 in 0.1 N HCI dissolution environment compared to the reference (Crestor®) dissolution profile.

2. As claimed in claim 1, alkali metals of carbonates or bicarbonate are selected from Na or K.

3. As claimed in claim 1, earth alkali metals of carbonates or bicarbonates are selected from Mg or Ca.

4. As claimed in claim 2, alkali metal carbonates are Na₂CO₃ anhydrous or K₂CO₃ anhydrous or mixture of thereof.

5. As claimed in claim 3, earth alkali metal carbonates are CaCO₃ or MgCO₃ or mixture of thereof.

6. As claimed in claim 1, pharmaceutical composition is oral dosage form.

7. As claimed in claim 6 oral dosage form is tablet.

8. As claimed in claim 1, pharmaceutical composition exhibits a dissolution profile as less than or equal to 85% of the total amount of rosuvastatin calcium is released in 30 minutes after combining the tablet with 900 ml of a dissolution medium at 37° C.±0.5° C. through using of USP method 1 (basket) and basket speed is 100 rpm.

9. As claimed in claim 1, pharmaceutical composition has a dissolution profile as rosuvastatin calcium is released in the range of 15% to 25% in 5 minutes, 40% to 50% in 10 minutes, 60% to 70% in 15 minutes, 70% to 80% in 20 minutes, 80% to 90% in 30 minutes, 85% to 95% in 45 minutes and 90 to 100% in 60 minutes after association of 900 ml of a dissolution medium at 37° C.±0.5° C. and USP method 1 (basket) and basket speed is 100 rpm.

10. A pharmaceutical composition comprising rosuvastatin or its pharmaceutically acceptable salt and alkali or earth alkali metal-carbonate-bicarbonate or a mixture thereof characterizing in that alkali or earth alkali metal-carbonate-bicarbonate or a mixture of thereof is used in the range of 0.5% to 2% by weight of pharmaceutical composition to provide a dissolution profile with a similarity factor (f2) of at least 50 to 100 in 0.1 N HCI dissolution environment compared to the reference (Crestor ®) dissolution profile.

11. As claimed in claim 10, pharmaceutical composition is oral dosage form.

12. As claimed in claim 11 oral dosage form is tablet.

13. As claimed in claim 10, pharmaceutical composition exhibits a dissolution profile as less than or equal to 85% of the total amount of rosuvastatin calcium is released in 30 minutes after combining the tablet with 900 ml of a dissolution medium at 37° C.±0.5° C. through using of USP method 1 (basket) and basket speed is 100 rpm.

14. As claimed in claim 10, pharmaceutical composition has a dissolution profile as rosuvastatin calcium is released in the range of 15% to 25% in 5 minutes, 40% to 50% in 10 minutes, 60% to 70% in 15 minutes, 70% to 80% in 20 minutes, 80% to 90% in 30 minutes, 85% to 95% in 45 minutes and 90 to 100% in 60 minutes after association of 900 ml of a dissolution medium at 37° C.±0.5° C. and USP method 1 (basket) and basket speed is 100 rpm.
